(19) United States
(12) Patent Application Publication
(10) Pub. No.: US 2020/0385667 A1
(43) Pub. Date: Dec. 10, 2020

(54) CELL CULTURE MONITORING DEVICE AND CULTURE MONITORING METHOD

(71) Applicant: BOE TECHNOLOGY GROUP CO., LTD., BEIJING (CN)

(72) Inventor: Shi ZHOU, Beijing (CN)

(21) Appl. No.: 16/479,704

(22) PCT Filed: Sep. 27, 2018
(86) PCT No.: PCT/CN2018/107841

§ 371 Date: Jul. 22, 2019

(30) Foreign Application Priority Data
Jan. 30, 2018 (CN) 201810089834.2

(51) Int. Cl. C12M 1/34; C12M 1/00; C12M 3/06; C12M 3/00; B01L 3/00

(52) U.S. Cl. CPC C12M 41/46; C12M 41/14; B01L 3/545; C12M 23/48; C12M 23/22; C12M 27/16

(57) ABSTRACT

Embodiments of the present disclosure relate to a cell culture monitoring device and a culture monitoring method. The cell culture monitoring device includes: a culture vessel configured to receive a culture medium of a cell sheet; and an image acquisition apparatus configured to acquire an image of the cell sheet in the culture vessel to monitor growth status of the cell sheet.

### CROSS-REFERENCE TO RELATED APPLICATIONS

This application is a U.S. National Stage Application under 35 U.S.C. § 371 of International Patent Application No. PCT/CN2018/107841, filed on Sep. 27, 2018, which claims priority to Chinese Patent Application No. 201810089834.2, filed on Jan. 30, 2018, the entire contents of which are herein incorporated by reference in entirety.

### TECHNICAL FIELD

The present disclosure relates to the field of tissue engineering, in particular to a cell culture monitoring device and a culture monitoring method.

### BACKGROUND

Temperature-responsive culture dishes are mainly applied in Cell Sheet Technology (CST) to harvest cells. CST avoids the use of proteases for cell treatment, thus preserving extracellular matrix secreted by cells and related proteins and factors during culture, and collecting cells in a complete membrane structure. Cell sheet are a research hotspot in the field of tissue engineering in recent years, and have been widely applied in the treatment of skin, cornea, heart and periodontal diseases.

### SUMMARY

In an aspect of the present disclosure, a cell culture monitoring is provided, including:

a culture vessel configured to receive a culture medium of a cell sheet; and

an image acquisition apparatus configured to acquire an image of the cell sheet in the culture vessel to monitor growth status of the cell sheet.

In some embodiments, the bottom of the culture vessel includes:

a temperature-sensitive layer configured to carry the culture medium; and

a luminescent layer, located on a side of the temperature-sensitive layer away from the cell sheet, and configured to provide a backlight for image acquiring of the cell sheet by luminescence.

In some embodiments, a transparent heat-insulation layer is further arranged between the temperature-sensitive layer and the luminescent layer, and configured to at least partially reduce heat transferred between the temperature-sensitive layer and the luminescent layer.

In some embodiments, a cold light source is located inside the luminescent layer or adjacent to an outside of the luminescent layer.

In some embodiments, the bottom of the culture vessel further includes:

a wiring layer on a side of the luminescent layer away from the cell sheet.

In some embodiments, further including:

a temperature adjusting element configured to adjust an internal temperature of the culture vessel.

In some embodiments, a shading structure is arranged outside the culture vessel.

In some embodiments, the temperature adjusting element includes a plurality of semiconductor temperature controllers arranged along an outer wall of the culture vessel, the plurality of semiconductor temperature controllers being arranged to form a shading structure.

In some embodiments, the culture vessel is within an incubator.

In some embodiments, one or more space layers are arranged in the incubator, a bracket configured to support a plurality of the culture vessels is arranged in the space layer, and the plurality of culture vessels are fixedly or detachably mounted on the bracket.

In some embodiments, an integral or detachable positioning structure is arranged outside the culture vessel, the positioning structure is mating with the bracket and movable relative to the bracket.

In some embodiments, further including:

a guiding mechanism arranged above the culture vessel; and

a driving mechanism configured to drive the image acquisition apparatus to move on the guiding mechanism so as to adjust an image acquisition position among the plurality of the culture vessels.

In some embodiments, the guiding mechanism includes: a parallel paired first rails and a second rail arranged between the paired first rails; the driving mechanism includes:

a first driving mechanism, arranged between the paired first rails and the second rail, and configured to drive the second rail to move relative to the paired first rails along an extending direction of the first rails; and

a second driving mechanism, arranged between the second rail and the image acquisition apparatus, and configured to drive the image acquisition apparatus to move relative to the second rail along an extending direction of the second rail.

In some embodiments, further including at least one of the following apparatuses:

a control apparatus configured to control external environmental parameters of the culture vessel; or

an image processing apparatus configured to process the image of the cell sheet acquired by the image acquisition apparatus to obtain the growth status of the cell sheet.

In some embodiments, the control apparatus is connected to the image processing apparatus and configured to adjust the external environmental parameters of the culture vessel according to the growth status of the cell sheet.

In another aspect of the present disclosure, a culture monitoring method based on the aforementioned cell culture monitoring device is provided, including:

putting a culture medium of a cell sheet into the culture vessel so that the cell sheet grow in the culture medium; and

acquiring an image of the cell sheet in the culture vessel by the image acquisition apparatus during growth of the cell sheet to monitor growth status of the cell sheet.

In some embodiments, the cell culture monitoring device includes a guiding mechanism and a driving mechanism arranged above the culture vessel; the culture monitoring method further includes:

controlling the driving mechanism to drive the image acquisition apparatus to move on the guiding mechanism so as to adjust the image acquisition apparatus to move to an image acquisition position corresponding to the culture vessel; and

controlling the image acquisition apparatus to perform image acquisition, and controlling the driving mechanism after completing the acquisition to drive the image acquisition apparatus to move to an image acquisition position corresponding to another culture vessel to continue image acquisition.

### BRIEF DESCRIPTION OF THE DRAWINGS

The accompanying drawings, which are part of the specification, describe the embodiments of the present disclosure and, together with the specification, are used to explain the principle of the present disclosure.

With reference to the drawings, the present disclosure may be more clearly understood according to the following detailed description, in which:

FIG. 1 is a schematic diagram of a structure of the cell culture monitoring device according to some embodiments of the present disclosure;

FIG. 2 is a schematic top view of a culture vessel of the cell culture monitoring device according to some embodiments of the present disclosure;

FIG. 3 is a schematic diagram of a structure of the culture vessel of the cell culture monitoring device according to some embodiments of the present disclosure;

FIG. 4 is a schematic diagram of a bottom structure of the culture vessel of the cell culture monitoring device according to some embodiments of the present disclosure;

FIG. 5 is a schematic diagram of a bottom structure of the culture vessel of the cell culture monitoring device according to some other embodiments of the present disclosure;

FIG. 6 is a schematic diagram of an external structure of the culture vessel of the cell culture monitoring device according to some embodiments of the present disclosure;

FIG. 7 is a schematic diagram of a structure of the cell culture monitoring device according to some other embodiments of the present disclosure;

FIG. 8 is a schematic diagram showing that a positioning structure mates with a bracket in the cell culture monitoring device according to some other embodiments of the present disclosure;

FIG. 9 is a schematic diagram showing that a guiding structure mates with a driving structure in the cell culture monitoring device according to some embodiments of the present disclosure;

FIG. 10 is a schematic diagram of a process of the culture monitoring method according to some embodiments of the present disclosure;

FIG. 11 is a schematic diagram of a process of the culture monitoring method according to some other embodiments of the present disclosure.

It should be understood that the size of each part shown in the drawings is not drawn according to an actual proportional relation. Moreover, the same or similar reference signs denote the same or similar components.

### DETAILED DESCRIPTION

Various exemplary embodiments of the present disclosure are now described in detail with reference to the drawings. The descriptions of the exemplary embodiments are only illustrative, and by no means as any limitation to the present disclosure and an application or use thereof. The present disclosure can be implemented in many different forms and is not limited to the embodiments described herein. These embodiments are provided to make the present disclosure thorough and complete, and to fully express the scope of the present disclosure to those skilled in the art. It should be noted that, unless otherwise specified, relative arrangements of components and steps described in these embodiments are only exemplary, but not restrictive.

The terms “first”, “second”, and similar terms used in the present disclosure do not denote any order, quantity or importance, but are used to distinguish different parts. The words “comprise” or “include” and the like mean that the elements preceding the words cover the elements listed after the words, and do not exclude other elements. “Upper”, “lower”, “left”, “right” and the like are only used to indicate the relative positional relationship, and when the absolute positions of the described objects are changed, the relative positional relationship may also be changed accordingly.

In the present disclosure, when a specific device is located between the first device and the second device, an intermediate device may or may not exist between the specific device and the first device or the second device. When the specific device is connected to other device, the specific device may be directly connected to the other device without an intermediate device, or indirectly connected to the other device by an intermediate device.

All terms (including technical or scientific terms) used in the present disclosure have the same meanings as understood by those of ordinary skill in the art to which the present disclosure belongs, unless specifically defined otherwise. It should also be understood that the terms defined in, for example, a general dictionary should be interpreted as having the meanings consistent with their meanings in the contexts of related technologies, and should not be interpreted with idealized or extremely formal meanings, unless explicitly defined herein.

Technologies, methods and devices known by those of ordinary skill in related technologies may not be discussed in detail, but in appropriate situations, the technologies, methods and devices should be regarded as part of the specification.

In the technologies known to the inventor, in order to measure the culture of cells in a cell culture apparatus, the culture status of the cells is indirectly detected by sampling a culture solution in the cell culture apparatus and detecting changes in indicators of the culture solution. The inventor has found through research that such related technologies applied to cell culture monitoring of a cell sheet and the like do not consider the specificity of the cell sheet, and it is difficult to effectively detect indicators such as area, thickness, uniformity and flatness of the cell sheet.

Considering the demand for cell culture of a cell sheet and the like, it is difficult to use the monitoring schemes of the related cell culture apparatuses for monitoring one or more of area, thickness, uniformity, flatness and the like. In view of this, in order to meet the status monitoring requirements of cells including cell sheet and the like during culture, the present disclosure provides an implementation structure and principle of some embodiments of a cell culture monitoring device.

In the following description, the cell culture includes culture of single-layer or multi-layer cell sheet. The cultured cells may include, for example, human cells isolated from human and various animal cells isolated from mouse, rat, guinea pig, hamster, chicken, rabbit, pig, sheep, cattle, horse, dog, cat, monkey, etc. The types of cells may include, for example, keratinocytes, splenocytes, neurocytes, glial cells, pancreatic β cells, mesangial cells, epidermal cells, epithelial cells (corneal epithelial cells, oral mucosal epithelial cells, amniotic epithelial cells, etc.), endothelial cells (vascular endothelial cells, corneal endothelial cells, etc.), fibroblasts, parenchymal cells (hepatocytes, corneal parenchymal cells, etc.), muscle cells including smooth muscle cells such as vascular smooth muscle cells, fat cells, synovial cells, cartilage cells, chondrocytes, osteoblasts, osteoclasts, mammary gland cell, hepatocytes, periosteal-derived cells or mesenchymal cells, or precursor cells of the aforementioned cells. The cells may also include stem cells such as embryonic stem cells (ESCs) and mesenchymal stem cells (MSCs) or cancer cells.

In the following description, the biological source of cells may be homologous, heterologous or the same individual as long as it can be cultured at the cellular level. That is to say, these cells may be either allogenic cells or xenogenic cells. These cells may also be of the same type and different type, for example, the same type of cells of the same animal, different types of cells of the same animal, the same type of cells of heterologous animals, different types of cells of heterologous animals, the same type of cells of the same individual, different types of cells of the same individual, etc.

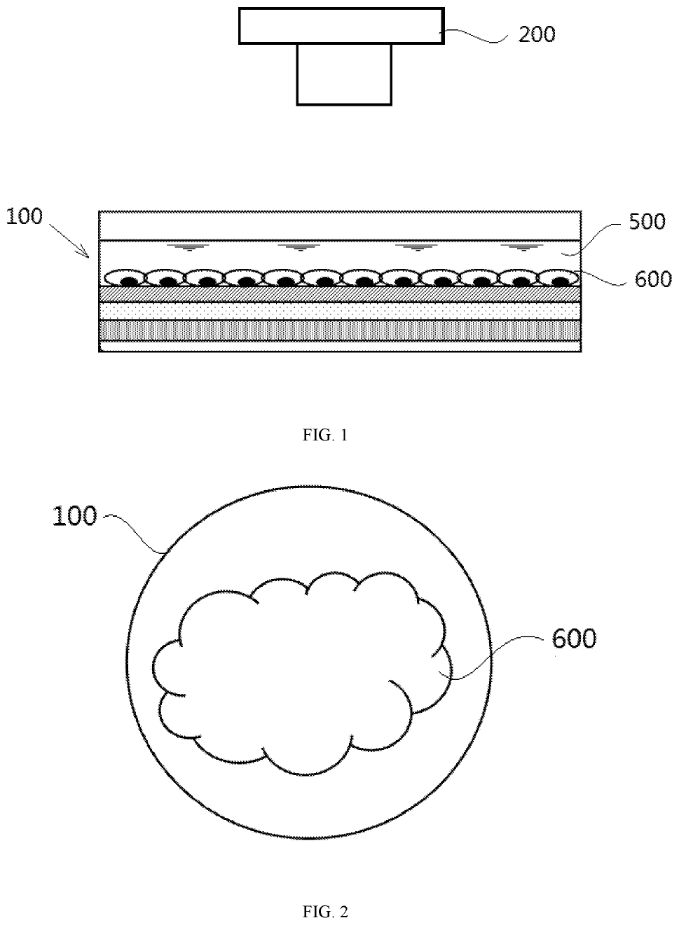

FIG. 1 is a schematic diagram of a structure of the cell culture monitoring device according to some embodiments of the present disclosure. Referring FIG. 1 and the top view of a culture vessel in FIG. 2, the cell culture monitoring device according to the present embodiment includes:

a culture vessel 100 configured to receive a culture medium 500 of a cell sheet 600; and

an image acquisition apparatus 200 configured to acquire an image of the cell sheet 600 in the culture vessel 100 to monitor growth status of the cell sheet 600.

In FIG. 1, the culture vessel 100 has a receiving space capable of receiving the culture medium 500 and the cell sheet 600, and provides a growth space for the cell sheet 600. The culture medium 500 is capable of providing nutrients to the cell sheet 600, so that cell sheet 600 can grow from single cells or cell colonies to the cell sheet 600 having certain area and thickness.

It is easy to understand that the composition and culture condition of the culture medium can be adjusted or changed according to the difference of cells to be cultured. For example, an appropriate culture medium and culture condition can be selected and designed according to the guidelines of Cold Spring Harbor Protocols (CSH Protocols).

The image acquisition apparatus 200 may be various types of imaging elements for shooting and imaging, e.g., cameras based on CCD or CMOS imaging, imagers based on infrared imaging or infrared thermal imaging, etc. The image acquisition apparatus 200 is capable of acquiring an image of the cell sheet in the culture vessel 100 in accordance with a preset time interval, on command or in real time. The image acquired by the image acquisition apparatus 200 may reflect the growth status of the cell sheet 600 in different phases. Moreover, important indicator data of the cell sheet 600, such as area, thickness, uniformity, flatness and the like, may be further obtained by analyzing and processing the image, thereby meeting the status monitoring requirement in the cell sheet culture process. When the cell sheet 600 are monitored to reach the required indicators of area and the like, the cell sheet 600 may be collected in time by a collection tool.

For example, when an image of the cell sheet 600 in the culture vessel 100 shown in FIG. 2 is obtained, the cover area of the cell sheet 600 in the image is measured. For another example, the thickness of the cell sheet 600 is determined by measuring the transmittance of the cell sheet 600 in the image and comparing with a preset standard value. Alternatively, the uniformity and/or flatness of the cell sheet 600 are determined by measuring a thermal distribution diagram and/or an infrared diagram of the cell sheet 600 in the image.

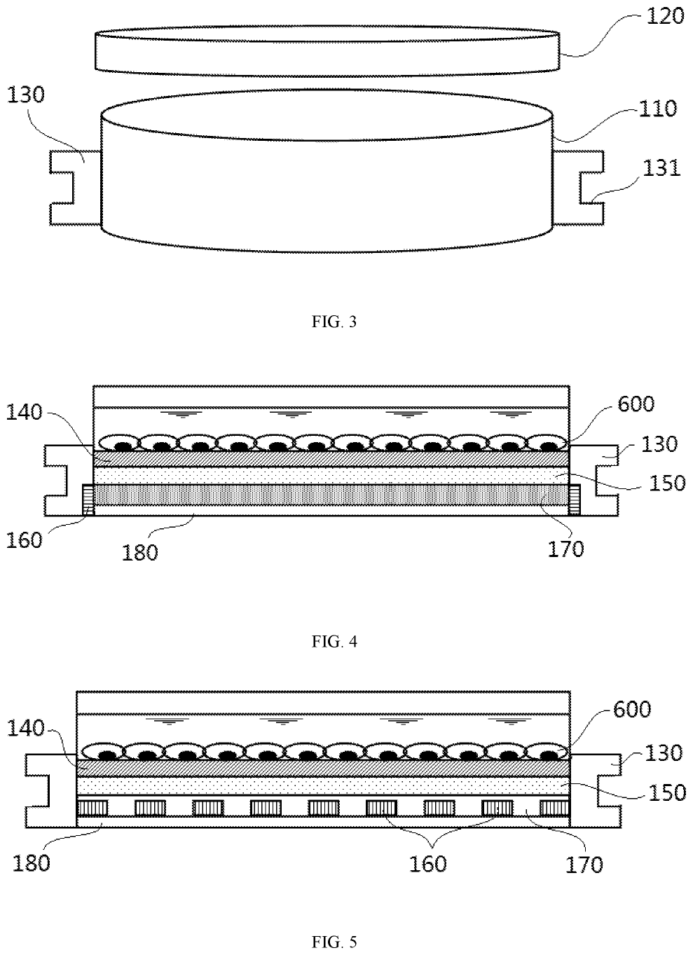

FIG. 3 is a schematic diagram of a structure of the culture vessel of the cell culture monitoring device according to some embodiments of the present disclosure. In FIG. 3, the culture vessel 100 includes a culture vessel body 110 and a cover 120 capable of covering the top of the culture vessel body 110 to close the internal space of the culture vessel body 110. The inner wall and the bottom of the culture vessel body 110 enclose a receiving space for the culture medium 500 and the cell sheet 600.

An integral or detachable positioning structure 130 may be arranged outside the culture vessel 100. Referring to FIG. 3, in some embodiments, the positioning structure 130 may be fixedly arranged outside the outer wall of the culture vessel body 110 to facilitate the position setting and adjustment of the culture vessel 110. In some other embodiments, to facilitate position adjustment of the positioning structure 130 relative to a guiding apparatus (e.g., a rail, etc.), a recessed structure 131 mating with the guiding apparatus may be arranged on the positioning structure 130.

For harvesting and collection of a cell sheet, a temperature-sensitive material is commonly used in related technologies as a basal layer (i.e., a temperature-sensitive layer) to carry a cell sheet, and such material may include POLY(N-isopropyl acrylamide) (PIPAAm), a complex of PIPAAm and methacrylic acid, lysine short peptide A6K, etc. The temperature-sensitive material is very sensitive to temperature. When the temperature sensed by the temperature-sensitive material reaches a specific condition, e.g., rises/falls to a specific temperature, the cell sheet change from a state close to the temperature-sensitive material to an easily detachable state, thereby facilitating harvesting and collection of the cell sheet.

In the technologies known to the inventor, some cell culture monitoring schemes use a light receiving portion of a measuring unit to receive light that is emitted by a light emitting portion and passing through the culture solution in a cell culture apparatus, and determines the culture status of cells according to the received light. When such cell culture monitoring schemes are applied to the culture monitoring of a cell sheet, the heat of a light source used by the light emitting portion may cause local overheating to damage the cell sheet, and may cause adverse effects on the temperature-sensitive material attached to the cell sheet, so that the cell sheet are very difficult to monitor.

In order to obtain high-quality images when the image acquisition apparatus 200 performs image acquisition, and to reduce the adverse effects on the temperature-sensitive material, referring to the bottom structure of the culture vessel shown in FIG. 4, the bottom of the culture vessel 100 includes a temperature-sensitive layer 140 and a luminescent layer 170, the luminescent layer 170 being on the side of the temperature-sensitive layer 140 away from the cell sheet 600. The temperature-sensitive layer 140 is configured to carry the culture medium 500. The luminescent layer 170 is configured to provide a backlight for image acquiring of the cell sheet 600 by luminescence (e.g., based on luminescence emitted by a cold light source 160) to ensure the quality of image acquisition.

The backlight provided by the luminescent layer 170 is luminescence, and the luminescence emitting process does not generate significant heat, such as fluorescence, phosphorescence and bacterial light, so little heat is emitted, the temperature of the temperature-sensitive layer 140 is less affected, and local overheating is unlikely to occur.

Alternatively, the luminescent layer 170 may be provided with light dispersing particles capable of dispersing the luminescence emitted by the cold light source 160 into the entire luminescent layer 170, thereby providing a more uniform backlight effect, and then improving the quality of image acquisition.

Alternatively, the cold light source 160 may emit luminescence based on the principles of photoluminescence, cathode ray luminescence or high-energy particle luminescence. In some embodiments of the present disclosure, the cold light source 160 may be a luminescent diode, luminescence rays, a fluorescent plate or a luminescent sheet, etc.

Referring to FIG. 4, in some embodiments, the cold light source 160 may be arranged adjacent to the outside of the luminescent layer 170. For example, in an embodiment in which an integral or detachable positioning structure 130 is arranged outside the culture vessel 100, the cold light source 160 may be arranged on the positioning structure 130 adjacent to the outside of the luminescent layer 170. The cold light source 160 is arranged outside the luminescence layer 170, which can further reduce the influence of heat of the cold light source 160 on the temperature-sensitive layer 140.

The position of the cold light source 160 is not limited thereto. FIG. 5 is a schematic diagram of a bottom structure of the cell culture monitoring device according to some other embodiments of the present disclosure. Referring to FIG. 5, the cold light source 160 may be arranged inside the luminescent layer 170 such that the structure of the culture vessel 100 is more compact. In some embodiments, a single cold light source 160 may be arranged inside the luminescent layer 170. In some other embodiments, a plurality of granular or elongated cold light sources 160 may be arranged inside the luminescent layer 170 and arranged randomly or in a predetermined array.

Referring to FIG. 4 and FIG. 5, in some embodiments, a transparent heat-insulation layer 150 is further arranged between the temperature-sensitive layer 140 and the luminescent layer 170. The transparent heat-insulation layer 150 may be configured to at least partially reduce heat transferred between the temperature-sensitive layer 140 and the luminescent layer 170 so as to further reduce the adverse effects of heat of the luminescent layer 170 on the temperature-sensitive layer 140. The transparent heat-insulation layer 150 may be made of a heat-insulation material that at least partially transmits the luminescence of the luminescent layer 170, e.g., glass or plastic that can transmit light.

Still referring to FIG. 4 and FIG. 5, in some embodiments, the bottom of the culture vessel 100 further includes a wiring layer 180. The wiring layer 180 is on the side of the luminescent layer 170 away from the cell sheet 600. The wiring layer 180 is arranged at the bottom of the culture vessel 100, so that the structure of the culture vessel 100 is more compact, and an external circuit is convenient to arrange.

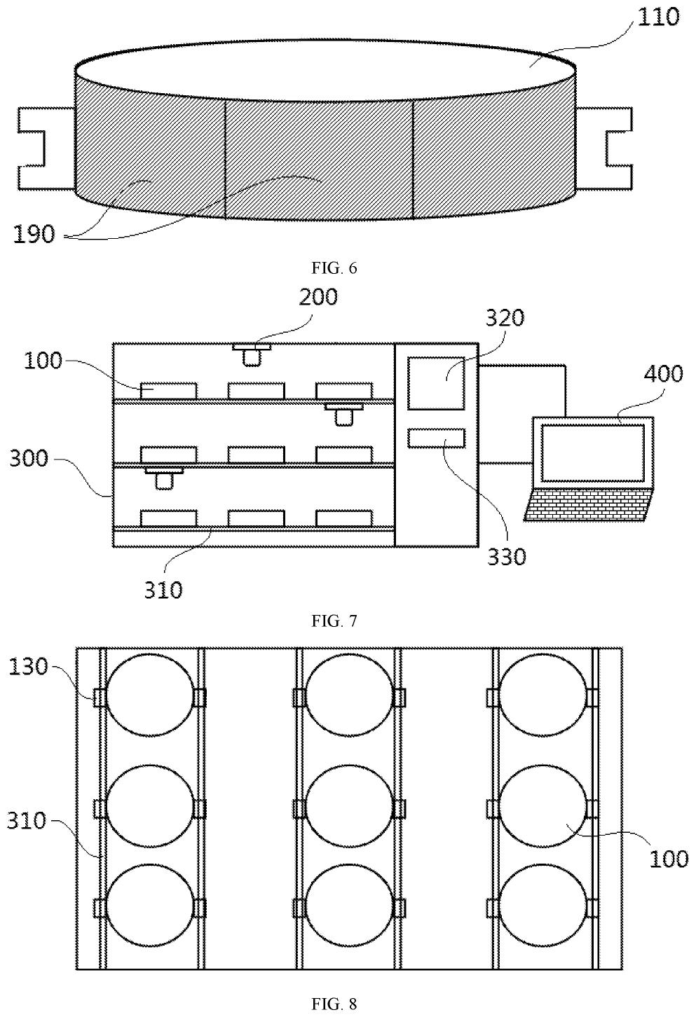

FIG. 6 is a schematic diagram of an external structure of the culture vessel of the cell culture monitoring device according to some embodiments of the present disclosure. In FIG. 6, a plurality of semiconductor temperature controllers 190 are closely arranged on the outer wall of the culture vessel 100. These semiconductor temperature controllers 190 may be used as a temperature adjusting element to adjust the internal temperature of the culture vessel 100 to achieve finer temperature control during culture of the cell sheet 600, thereby ensuring stable growth of the cell sheet 600. In addition, the temperature adjusting element may also stabilize the temperature-sensitive layer 140 during growth of the cell sheet 600. When the cell sheet 600 need to be collected, the temperature adjusting element may also adjust the internal temperature of the culture vessel 100 to a temperature range in which the temperature-sensitive layer 140 is easily isolated from the cell sheet 600, thereby reducing the difficulty in collecting the cell sheet 600.

The temperature adjusting element are not limited to the semiconductor temperature controllers 190 shown in FIG. 6. In some other embodiments, the temperature adjusting elements may also include electronic temperature controllers or vapor temperature controllers or the like. In terms of setting positions, the temperature adjusting element in some embodiments may be arranged inside the culture vessel 100, or outside the culture vessel 100 without fitting closely to the outer wall of the culture vessel 100, etc.

In order to prevent luminescence leakage or external light entry into the culture vessel to affect the quality of acquired images, a shading structure is arranged outside the culture vessel 100 in some embodiments. For example, FIG. 6 shows that a plurality of semiconductor temperature controllers 190 are arranged along the outer wall of the culture vessel 100 to form a shading structure. The semiconductor material is non-transparent with respect to visible light, and can form a shading structure, thereby achieving faster temperature adjustment and limiting leakage of internal luminescence or entry of external light.

FIG. 7 is a schematic diagram of a structure of the cell culture monitoring device according to some other embodiments of the present disclosure. In some other embodiments shown in FIG. 7, the culture vessel 100 is arranged inside an incubator 300. The incubator 300 is capable of providing an external environment suitable for the growth of a cell sheet 600 to increase the controllability and reliability of the external environment for the growth of the cell sheet 600. The incubator 300 is very convenient in many aspects such as arrangement, setting and use, and is advantageous for being used by an operator.

Referring to FIG. 7, in some embodiments, one or more space layers are arranged in the incubator 300. A bracket 310 configured to support a plurality of the culture vessels 100 is arranged in the space layers, and the plurality of culture vessels 100 are fixedly or detachably mounted on the bracket 310, thereby meeting the culture and monitoring requirements of batches of cell sheets, benefiting large-scale and industrial culture of the cell sheets, and reducing the production cost of the cell sheets.

In order to control external environmental parameters of the culture vessel 100 (e.g., set operating parameters of the incubator 300, such as temperature, operating time, operating parameter, on and off), a control apparatus capable of controlling the external environmental parameters of the culture vessel 100 may be included in some other embodiments. For example, in FIG. 7, an operation panel 330 on the incubator 300 receives the input of external environmental parameters, and a display screen 320 displays related parameters and monitoring data.

In order to process the images of the cell sheet 600, an image processing apparatus may also be included in some other embodiments. The image processing apparatus may be configured to process the images of the cell sheet 600 acquired by the image acquisition apparatus 200 to obtain the growth status of the cell sheet 600. In some embodiments, the control apparatus is connected to the image processing apparatus and configured to adjust the external environmental parameters of the culture vessel according to the growth status of the cell sheet. For example, in FIG. 7, a computer 400 is connected to the image acquisition apparatus 200 in the incubator 300, and processes the images of the cell sheet 600 acquired by the image acquisition apparatus 200 to obtain important indicator data of the cell sheet 600, such as area, thickness, uniformity, flatness and the like.

For example, the control apparatus, the image processing apparatus, and the like may be implemented by a processor having data processing capability and/or program execution capability, such as a central processing unit (CPU) or a field programmable logic array (FPGA) or a single chip microcomputer (MCU) or a digital signal processor (DSP) or an application-specific integrated circuit (ASIC).

For example, the control apparatus and the image processing apparatus may be integrated in the same processor or separately implemented by different processors.

For example, the control apparatus may be connected to the image processing apparatus.

For example, the connection may be implemented by a wireless network, a wired network, and/or any combination of a wireless network and a wired network, etc. The network may include a local area network, the Internet, a telecommunications network, an Internet of things based on the Internet and/or telecommunications network, and/or any combination of the above networks, etc. The wired network may communicate by, for example, twisted pair, coaxial cable or optical fiber transmission. The wireless network may communicate by, for example, a mobile communication network, Bluetooth, Zigbee or Wi-Fi.

FIG. 8 is a schematic diagram showing that a positioning structure mates with a bracket in the cell culture monitoring device according to some other embodiments of the present disclosure. Referring to FIG. 3 and FIG. 8, in some embodiments, an integral or detachable positioning structure 130 is arranged outside the culture vessel 100, the positioning structure 130 is mating with the bracket 310 and movable relative to the bracket 310. The bracket 310 in the form of an elongated rod shown in FIG. 8 may be in guiding fit with a recessed structure 131 of the positioning structure 130 in addition to supporting the culture vessel 100, so that the arrangement and position adjustment of the culture vessel 100 on the bracket 310 are more convenient.

FIG. 7 and FIG. 8 illustrate the arrangement of multiple layers and sets of culture vessels 100 in the incubator 300 in some embodiments. One or more image acquisition apparatuses 200 may be arranged on each layer of the incubator 300. The image acquisition apparatuses 200 may move to image acquisition positions corresponding to different culture vessels 100 for image acquisition. In some other embodiments, the image acquisition apparatuses 200 may also be arranged outside the incubator 300 to simplify the structure of the incubator 300 and reduce the space of the incubator 300.

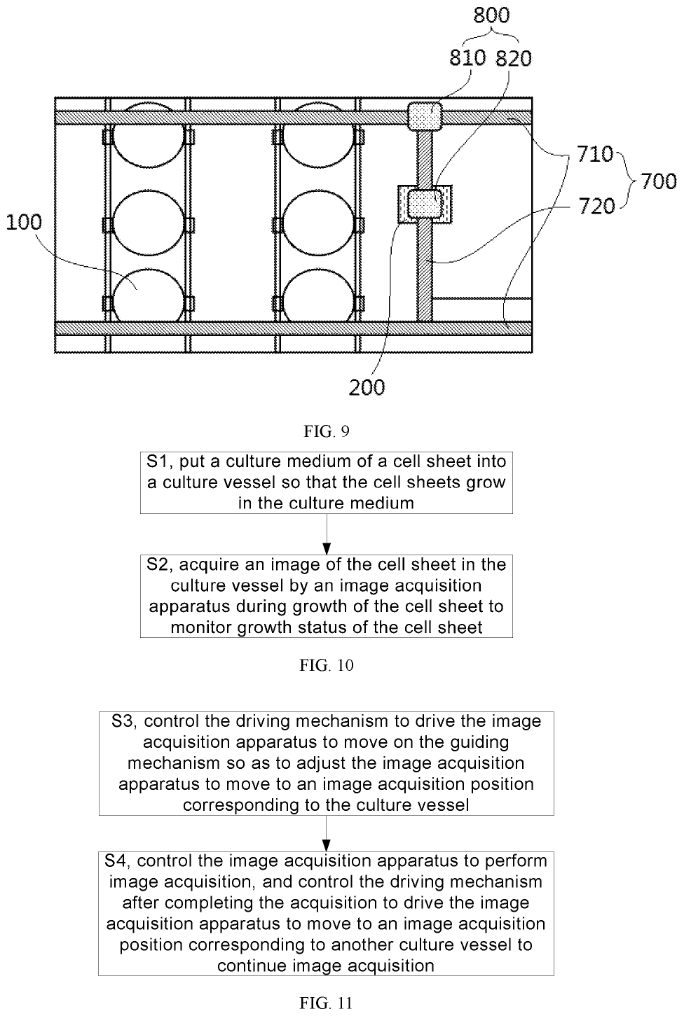

In some embodiments, the image acquisition positions of the image acquisition apparatuses 200 may be manually adjusted by an operator. In some other embodiments, automatic adjustment may also be employed to save manpower and improve efficiency. FIG. 9 is a schematic diagram showing that a guiding structure mates with a driving structure in the cell culture monitoring device according to some embodiments of the present disclosure. Referring to FIG. 9, in some embodiments, the cell culture monitoring device further includes a guiding mechanism 700 and a driving mechanism 800. The guiding mechanism 700 is arranged above the culture vessels 100, and the driving mechanism 800 is configured to drive the image acquisition apparatus 200 to move on the guiding mechanism 700, thereby adjusting the image acquisition position of the image acquisition apparatus 200 among the plurality of culture vessels 100. For the plurality of culture vessels 100, these examples allow a smaller number of image acquisition apparatuses to acquire images in different time intervals by means of position adjustment, thereby reducing the use of image acquisition apparatuses and lowering the product cost and energy consumption while meeting the monitoring requirements.

FIG. 9 shows an example of a mating structure of the guiding mechanism and a driving mechanism, which is simple and easy to implement. In FIG. 9, the guiding mechanism 700 includes a parallel paired first rails 710 and a second rail 720 between the paired first rails 710. The driving mechanism 800 includes a first driving mechanism 810 and a second driving mechanism 820. The first driving mechanism 810 is arranged between the paired first rails 710 and the second rail 720, and configured to drive the second rail 720 to move relative to the paired first rails 710 along an extending direction of the first rails 710. The second driving mechanism 820 is arranged between the second rail 720 and the image acquisition apparatus 200, and configured to drive the image acquisition apparatus 200 to move relative to the second rail 720 along an extending direction of the second rail 720. The first driving mechanism 810 and the second driving mechanism 820 may be various driving power components (e.g., motors, gas pumps or hydraulic pumps, etc.), and a transmission structure (e.g., a gear rack transmission structure, a gear train transmission structure or a pulley block transmission mechanism, etc.) may be selected as needed.

According to the requirements of image acquisition, when image acquisition is required for a certain culture vessel 100, the driving mechanism 800 may be controlled to drive the image acquisition apparatus 200 to move on the guiding mechanism 700, so that the image acquisition apparatus 200 moves to the image acquisition position corresponding to the culture vessel 100. For example, the first driving mechanism 810 may first drive the second rail 720 to move along the paired first rails 710 to the column where the culture vessel 100 is located, and the second driving mechanism 820 drives the image acquisition apparatus 200 to move to a position above the culture vessel 100 which is the image acquisition position corresponding to the culture vessel 100. In other examples, the driving steps of the first driving mechanism 810 and the second driving mechanism 820 may be reversed or performed simultaneously.

After the adjustment is in place, the image acquisition apparatus 200 may be controlled to perform image acquisition, and the driving mechanism 800 is controlled after completing the acquisition to drive the image acquisition apparatus 200 to move to an image acquisition position corresponding to another culture vessel 100 to continue image acquisition.

Various embodiments of the cell culture monitoring device according to the present disclosure have been described above. Based on any embodiment of the cell culture monitoring device described above, the present disclosure also provides corresponding embodiments of a culture monitoring method. FIG. 10 is a schematic diagram of a process of the culture monitoring method according to some embodiments of the present disclosure. In the present embodiments, the culture monitoring method may include:

Step S1, a culture medium 500 of a cell sheet 600 is put into a culture vessel 100 so that the cell sheet 600 grow in the culture medium 500;

Step S2, an image of the cell sheet 600 in the culture vessel 100 is acquired by the image acquisition apparatus during growth of the cell sheet 600 to monitor growth status of the cell sheet 600.

In the method embodiments, the culture medium 500 and single cells or cell colonies as the basis for the growth of a cell sheet 600 may be put into the culture vessel 100 by an operator manually or a tool, and then the image acquisition apparatus 200 is manually operated by the operator or automatically controlled by the control apparatus during the growth of the cell sheet 600 to acquire an image of the cell sheet 600 in the culture vessel 100.

Referring to FIG. 11, in some other cell culture monitoring device embodiments, a guiding mechanism 700 and a driving mechanism 800 arranged above the culture vessel 100 may be further included.

The corresponding culture monitoring method may further include:

Step S3, the driving mechanism 800 is controlled to drive the image acquisition apparatus 200 to move on the guiding mechanism 700 so as to adjust the image acquisition apparatus 200 to move to an image acquisition position corresponding to the culture vessel 100; and

Step S4, the image acquisition apparatus 200 is controlled to perform image acquisition, and the driving mechanism 800 is controlled after completing the acquisition to drive the image acquisition apparatus 200 to move to an image acquisition position corresponding to another culture vessel 100 to continue image acquisition.

In the present embodiment, the driving operation of the driving mechanism 800 and the image acquisition operation of the image acquisition apparatus 200 may be controlled by a single or different control apparatuses, and the control apparatuses may be implemented by a general-purpose or special-purpose computing device running control programs. Step S3 and step S4 may be embodied as step S2 of the foregoing method embodiment, or performed independently of step S2.

The multiple embodiments in the Description are described progressively, each embodiment focuses on difference from other embodiments, and the same or similar parts of each embodiment refer to each other. The whole and steps of the method embodiments correspond to the contents in the apparatus embodiments and therefore are briefly described, and reference may be made to the apparatus embodiments for the associated parts.

So far, various embodiments of the present disclosure are described in detail. In order to avoid obscuring the concept of the present disclosure, some details known in the art are not described. Those skilled in the art can fully understand how to implement the technical solutions disclosed herein according to the above description.

Although some specific embodiments of the present disclosure have been described in detail by way of examples, it should be understood by those skilled in the art that the above examples are merely for describing, rather than limiting the scope of the present disclosure. It should be appreciated by those skilled in the art that the above embodiments may be modified or some of the technical features may be substituted without departing from the scope and spirit of the present disclosure. The scope of the present disclosure is defined by the appended claims.

1. A cell culture monitoring device, comprising:
a culture vessel configured to receive a culture medium of a cell sheet; and
an image acquisition apparatus configured to acquire an image of the cell sheet in the culture vessel to monitor growth status of the cell sheet.

2. The cell culture monitoring device according to claim 1, wherein a bottom of the culture vessel comprises:
a temperature-sensitive layer configured to carry the culture medium; and
a luminescent layer, located on a side of the temperature-sensitive layer away from the cell sheet, and configured to provide a backlight for image acquiring of the cell sheet by luminescence.

3. The cell culture monitoring device according to claim 2, wherein a transparent heat-insulation layer is further arranged between the temperature-sensitive layer and the luminescent layer, and configured to at least partially reduce heat transferred between the temperature-sensitive layer and the luminescent layer.

4. The cell culture monitoring device according to claim 2, wherein a cold light source is located inside the luminescent layer or adjacent to an outside of the luminescent layer.

5. The cell culture monitoring device according to claim 2, wherein the bottom of the culture vessel further comprises:
a wiring layer on a side of the luminescent layer away from the cell sheet.

6. The cell culture monitoring device according to claim 1, further comprising:
a temperature adjusting element configured to adjust an internal temperature of the culture vessel.

7. The cell culture monitoring device according to claim 2, wherein a shading structure is arranged outside the culture vessel.

8. The cell culture monitoring device according to claim 6, wherein the temperature adjusting element comprises a plurality of semiconductor temperature controllers arranged along an outer wall of the culture vessel, the plurality of semiconductor temperature controllers being arranged to form a shading structure.

9. The cell culture monitoring device according to claim 1, wherein the culture vessel is within an incubator.

10. The cell culture monitoring device according to claim 9, wherein one or more space layers are arranged in the incubator, a bracket configured to support a plurality of culture vessels is arranged in the one or more space layers, and the plurality of culture vessels are fixedly or detachably mounted on the bracket.

11. The cell culture monitoring device according to claim 10, wherein an integral or detachable positioning structure is arranged outside the culture vessel, the positioning structure is mating with the bracket and movable relative to the bracket.

12. The cell culture monitoring device according to claim 1, further comprising:
a guiding mechanism arranged above the culture vessel; and
a driving mechanism configured to drive the image acquisition apparatus to move on the guiding mechanism so as to adjust an image acquisition position among the plurality of culture vessels.

13. The cell culture monitoring device according to claim 12, wherein:
the guiding mechanism comprises: a parallel paired first rails and a second rail arranged between the paired first rails;
the driving mechanism comprises:
a first driving mechanism, arranged between the paired first rails and the second rail, and configured to drive the second rail to move relative to the paired first rails along an extending direction of the paired first rails; and
a second driving mechanism, arranged between the second rail and the image acquisition apparatus, and configured to drive the image acquisition apparatus to move relative to the second rail along an extending direction of the second rail.

14. The cell culture monitoring device according to claim 1, further comprising at least one of the following apparatuses:
a control apparatus configured to control external environmental parameters of the culture vessel; or
an image processing apparatus configured to process the image of the cell sheet acquired by the image acquisition apparatus to obtain the growth status of the cell sheet.

15. The cell culture monitoring device according to claim 14, wherein the control apparatus is connected to the image processing apparatus and configured to adjust the external environmental parameters of the culture vessel according to the growth status of the cell sheet.

16. A culture monitoring method, comprising:
putting a culture medium of a cell sheet into a culture vessel so that the cell sheet grow in the culture medium; and
acquiring an image of the cell sheet in the culture vessel by an image acquisition apparatus during growth of the cell sheet to monitor growth status of the cell sheet.

17. The culture monitoring method according to claim 16, wherein the cell culture monitoring device comprises a guiding mechanism and a driving mechanism arranged above the culture vessel; the culture monitoring method further comprises:
controlling the driving mechanism to drive the image acquisition apparatus to move on the guiding mechanism so as to adjust the image acquisition apparatus to move to an image acquisition position corresponding to the culture vessel; and
controlling the image acquisition apparatus to perform image acquisition, and controlling the driving mechanism after completing the image acquisition to drive the image acquisition apparatus to move to an image acquisition position corresponding to another culture vessel to continue the image acquisition.
